(19) United States
(12) Patent Application Publication
(10) Pub. No.: US 2026/0227375 A1
(43) Pub. Date: Aug. 6, 2026

(54) SYSTEMS, METHODS, AND MEDIA FOR AUTOMATED MILK QUALITY MONITORING USING INLINE SENSORS

(71) Applicant: Technologies Holdings Corp., Houston, TX (US)

(72) Inventors: Damien Constantine, Riverview (CA); Steve Pretz, Westwood Hills, KS (US)

(73) Assignee: Technologies Holdings Corp., Houston, TX (US)

(21) Appl. No.: 19/044,917

(22) Filed: Feb. 4, 2025

(51) Int. Cl. G01N 33/06; A01J 5/01; A01J 5/013; G01N 21/39; G01N 33/68; G01N 35/00

(52) U.S. Cl. CPC G01N 33/06; A01J 5/01; A01J 5/0131; G01N 21/39; G01N 33/6803; G01N 35/0099

(57) ABSTRACT

A system for automated milk quality monitoring using inline sensors is provided, the system comprising: milking stations each associated with a respective milk line; inline sensors, each configured to measure milk composition information of milk flowing through one of the milk lines, wherein a first subset of the milk lines are equipped with one of the inline sensors and generate milk composition information for milk flowing through the first subset of milk lines during milking, and a second subset of the milk lines are not equipped with one of the inline sensors; and a processor configured to: receive, from each inline sensor, data indicative of milk composition information of milk flowing through that inline sensor; associate the received data with an animal; store milk composition information for the milk from the animal; and determine milk quality information using milk composition information for multiple milking sessions of the animal.

### CROSS-REFERENCE TO RELATED APPLICATIONS

N/A

### BACKGROUND

Currently, the general industry standard for determining milk quality being produced by cows on a dairy farm is testing based on standards promoted by the Dairy Heard Improvement Association (DHIA testing), and/or similar organizations. For example, DHIA testing generally includes projecting a cow's milk production by taking a sample of milk from each cow one time per month, and analyzing the milk in a laboratory setting to determine various properties of the milk, which results in milk production for most cows being measured only 10 times in a typical 305 lactation period. This practice is costly and labor intensive, and becomes increasingly costly and labor intensive as the number of cows at a dairy farm increases. Additionally, research indicates that milk composition differs from day to day, and varies throughout the day. Accordingly, even assuming that there are zero human errors in the collection and analysis of the milk samples collected once per month, the statistical value of the data is greatly diminished.

### SUMMARY

In accordance with some embodiments of the disclosed subject matter, a system for automated milk quality monitoring using inline sensors is provided, the system comprising: a plurality of milking stations, each associated with a respective milk line of a plurality of milk lines; a plurality of inline sensors, each configured to measure milk composition information of milk flowing through one of the plurality of milk lines, wherein a first subset of milk lines of the plurality of milk lines are equipped with a respective inline sensor of the plurality of inline sensors, such that milk composition information is generated for milk flowing through the first subset of milk lines during milking, and wherein a second subset of milk lines the plurality of milk lines are not equipped with inline an inline sensor configured to measure milk composition information of milk flowing through one of the second subset of milk lines, such that milk composition information is not generated for milk flowing through the second subset of milk lines during milking; one or more hardware processors configured to: receive, from each of the plurality of inline sensors, data indicative of milk composition information of milk flowing through that inline sensor during a milking session; associate the received data with a particular animal that produced the milk; store milk composition information for milk from the particular animal during the milking session; and determine milk quality information for the particular animal using stored milk composition information for a plurality of milking sessions over a predetermined period of time.

In some embodiments, the number of milk lines in the first subset of milk lines is less than the number of milk lines in the second subset of milk lines.

In some embodiments, each milk line of the plurality of milk lines is a member of the first subset of milk lines of the second subset of milk lines.

In some embodiments, each of the plurality of inline sensors comprises: a laser source configured to emit coherent light through milk flowing through the inline sensor; and a laser detector configured to generate spectral data indicative of the concentration of one or more milk components in the milk through which the coherent light passed.

In some embodiments, each of the plurality of inline sensors comprises: a contact sensor configured to determine a volumetric flow of milk through the sensor.

In some embodiments, the system further comprises: a plurality of inline volume sensors, each configured to measure volume flow of milk flowing through one of the plurality of milk lines, wherein each milk line in the first subset of milk lines is equipped with a respective inline volume sensor of the plurality of inline volume sensors, such that milk volume information is generated for milk flowing through the first subset of milk lines during milking.

In some embodiments, the one or more hardware processors are further configured to: receive data indicative of volumetric flow of milk flowing through a particular milk line during the milking session; determine the milk composition information for milk from a particular animal during a particular milking session based on the data indicative of milk composition information of milk flowing through the particular milk line during the milking session and the data indicative of volumetric flow of milk flowing through the particular milk line during the milking session.

In some embodiments, the plurality of milking stations comprise all of the milk stations of a milking operation, such that only a fraction of milking stations of the milking operation are equipped with inline sensors configured to measure milk composition information of milk during milking.

In some embodiments, the first subset of milk lines is about ten percent of the plurality of milking stations.

In some embodiments, the milking operation comprises a robotic system configured to autonomously attach milking devices to animals at the plurality of milking stations for milking.

In some embodiments, the stored milk composition information comprises fat content information for the milking session, protein content information for the milking session, and lactose content information for the milking session.

In some embodiments, the milk quality information is an aggregate value based on milk quality information for each of the plurality of milking sessions.

In some embodiments, the predetermined period of time is at least seven days.

In accordance with some embodiments of the disclosed subject matter, a method for automated milk quality monitoring using inline sensors is provided, the method comprising: receiving, from each of a plurality of inline sensors, data indicative of milk composition information of milk flowing through that inline sensor during a milking session, wherein each of the plurality of inline sensors is configured to measure milk composition information of milk flowing through one of a plurality of milk lines, which are each associated with a respective milking station of a plurality of milking stations, wherein a first subset of milk lines of the plurality of milk lines are equipped with a respective inline sensor of the plurality of inline sensors, such that milk composition information is generated for milk flowing through the first subset of milk lines during milking, and wherein a second subset of milk lines the plurality of milk lines are not equipped with inline an inline sensor configured to measure milk composition information of milk flowing through one of the second subset of milk lines, such that milk composition information is not generated for milk flowing through the second subset of milk lines during milking; associating the received data with a particular animal that produced the milk; storing milk composition information for milk from the particular animal during the milking session; and determining milk quality information for the particular animal using stored milk composition information for a plurality of milking sessions over a predetermined period of time.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1A shows an example of a system for automated milk quality monitoring using inline sensors in accordance with some embodiments of the disclosed subject matter.

FIG. 1B shows an example of a portion of a system for automated milk quality monitoring using inline sensors in accordance with some embodiments of the disclosed subject matter.

FIG. 2 shows an example of a system for analyzing milk quality using data from inline sensors in accordance with some embodiments of the disclosed subject matter.

FIG. 3 shows an example of hardware that can be used to implement a data source, a computing device, and a server in accordance with some embodiments of the disclosure.

FIG. 4 shows an example of a process for automated milk quality monitoring using inline sensors in accordance with some embodiments of the disclosed subject matter.

FIG. 5 shows an example flow for automated milk quality monitoring using inline sensors in accordance with some embodiments of the disclosed subject matter.

### DETAILED DESCRIPTION

As described above, the value of milk quality information collected using conventional sampling techniques is limited due to, in part, to the infrequency at which samples are taken, and the time at which each sample was taken. However, accurate milk quality data is critical for accurately calculating feeding efficiency, which can have a large impact on the monetary success of a dairy farm, as feed is typically the largest expense on a dairy farm. Additionally, accurate milk quality data is critical for identifying cows with genetics associated with desirable traits (e.g., that result in higher volume milk production, more consistent milk volume production, higher fat content, etc.).

In some embodiments, mechanisms described herein can collect milk quality information using inline sensors that measure characteristics of milk during milking as the milk flows from the dairy animal (e.g., the cow) to a milk storage tank. While installing such an inline sensor on every milk line (or even for every teat of each cow) would be expected to generate the most valuable data, such inline sensors are costly to purchase and install. In some embodiments, mechanisms described herein can facilitate collection of milk quality information for each cow in dairy operation with a significantly higher sampling frequency than conventional techniques that collect only limited samples. For example, in some embodiments, mechanisms described herein can use a quantity of sensors that is less than the number of milking stalls in a dairy operation, which can be used to statistically quantify milk quality over several milking sessions to a high level of confidence. Unlike conventional monthly sampling, mechanisms described herein can be used continuously, resulting in increased sampling per lactation period. For example, in a typical large herd installation, the number of samples taken can be expected to increase from 10 samples per lactation to about 60 samples per lactation when about 10% of milk stalls are monitored using the inline sensors. Additionally, these samples can be expected to be from various times of day, and mitigates the potential that the owner of the herd may attempt to manipulate data and/or mitigates common sources of human error during sampling (e.g., by mislabeling, mishandling, etc.). Additionally, such inline sensors can use techniques that measure electric and/or optical properties of the milk, rather than using reagents to measure chemical properties of the milk, without ongoing costs associated with replenishing reagents.

As described above, while there are benefits to installing one or more inline sensors for each milk line, there are also costs associated with installing additional sensors, such as a cost to purchase each sensor, a cost to install each sensor (e.g., which may involve electrical work to provide a source of electrical power to each inline sensor), a cost to monitor operation of the inline sensor (e.g., to determine whether data being generated by each inline sensor is relatively accurate), a cost to perform regular schedule maintenance on the sensor (e.g., which may be higher than costs associated with maintaining a milk line that is not provided with an inline sensor(s)), a cost to perform unscheduled maintenance (e.g., if the sensor malfunctions), etc. In such an example, while providing one or more inline sensors for each milk line to facilitate monitoring of milk characteristics for every milking session for every cow, purchasing, installing and maintaining that number of sensors may outweigh the benefits for many dairy owner and/or operators. Monitoring milk quality using inline sensors on only a fraction of milking sessions reduces the costs of monitoring, and also does not scale at a one to one cost with increases in the size of the herd.

In some embodiments, mechanisms described herein can facilitate more robust monitoring of milk quality for a herd of dairy livestock, at a reduced cost compared to a system that monitors every milking of every animal. Note that although mechanisms described herein are generally described in connection with milking cows, mechanisms described herein can be used in connection with other types of dairy livestock, such as goats, sheep, etc.

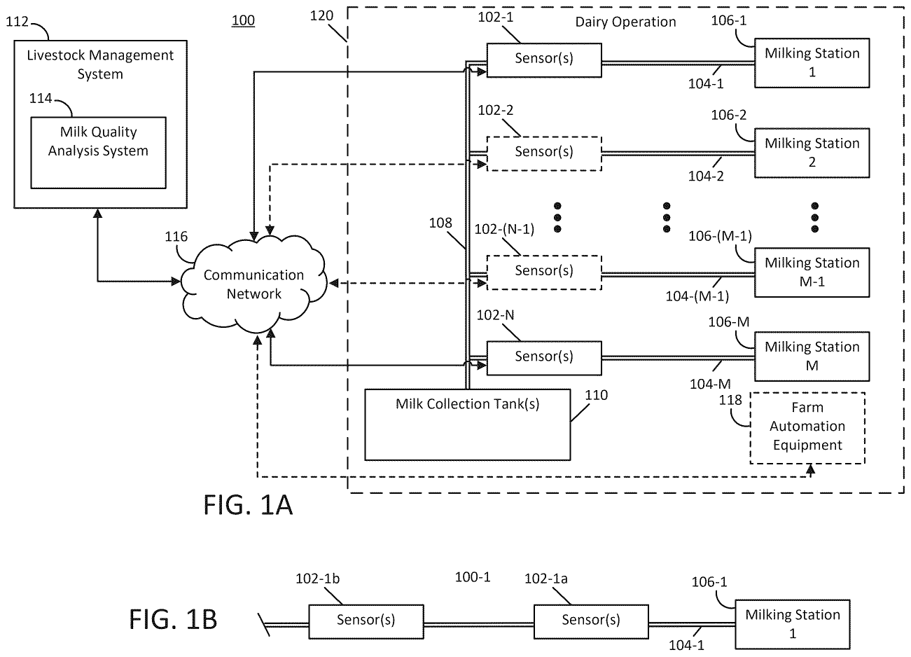

FIG. 1A shows an example of a system 100 for automated milk quality monitoring using inline sensors in accordance with some embodiments of the disclosed subject matter. As shown in FIG. 1A, system 100 can include a dairy operation 120 which can include any suitable number of milking stations 106 (e.g., M milking stations 106-1 to 106-M), which can each be configured to milk a dairy livestock animal, such as a dairy cow. For example, each milking station 106 can include, or otherwise be associated with, a milking device(s) that can be attached to the teats of a cow to automatically extract milk from the cow. In some embodiments, each milking station can be associated with a milk line 104 (e.g., M milk lines 104-1 to 104-M corresponding to milking stations 106-1 to 106-M) that can be used to direct milk toward one or more milk tanks 110 for collection. In the example of FIG. 1A, multiple milk lines 104 are connected to milk tank 110 via a bulk line 108. However, one or more of milk lines 104-1 to 104-M can be individually connected to milk tank 110, and/or one or groups of milk lines 104-1 to 104-M can each be connected to milk tank 110 via a respective bulk line. Note that although not shown, milk lines 104-1 to 104-M can be connected to multiple milk tanks (e.g., for storing milk with different characteristics) and/or to a waste disposal line (e.g., directed to a waste tank, a sewer line, etc.) via any suitable valves and/or other flow control devices.

In some embodiments, system 100 can include any suitable number of inline sensors 102 (e.g., a number N that is less than the total number of milking stations M), which can each be associated with a particular milking station of milking stations 106-1 to 106-M, and which are configured to analyze fluid flowing through a respective milk line 104-1 to 104-M associated with the associated milking station. For example, inline sensor(s) 102 can be integrated into the associated milk line, and/or can be retrofitted to an existing milk line. In some embodiments, each inline sensor 102 can include a sensor(s) that can be used to determine one or more characteristics of milk flowing through the associated milk line (e.g., inline sensor 102-1 can include a sensor(s) that can be used to analyze milk flowing through milk line 104-1, and can generate data that can be used to determine a characteristic(s) of the milk, as the milk flows through sensor 102-1).

In some embodiments, inline sensor 102 can be configured to generate data that can be used to determine any suitable characteristic(s) of the milk flowing through inline sensor 102. In some embodiments, inline sensor 102 can be configured to generate data that can be used to determine an amount and/or concentration of one or more components of milk, such as one or more main components (e.g., fat. protein, and/or lactose), one or more ancillary and/or trace components (e.g., metabolites and/or other analytes that are generally present in relatively small amounts, etc.), one or more potential contaminants (e.g., blood cells, bacteria, etc.), and/or one or more parameters indicative of an amount of milk flowing through, and/or that has flowed through, the inline sensor and/or milk line. For example, inline sensor 102 can be configured to generate data that can be used to determine milk composition information indicative of the amount (e.g., in grams) and/or concentration (e.g., weight per volume such as milligrams per deciliter (mg/dl), percent by weight, percent by volume, etc.) of main components of milk (e.g., components that are generally expected to make up at least 1% by weight of normal milk), such as fat content, protein content, lactose content, and/or water content of milk flowing through inline sensor 102. As another example, inline sensor 102 can be configured to generate data that can be used to determine milk composition information indicative of an estimated amount and/or concentration (e.g., cells per milliliter (cells/ml), milligrams per deciliter (mg/dl), parts per million, percent by weight, percent by volume, etc.) of ancillary and/or trace components of milk such as concentration of a urea-containing molecule (e.g., milk urea nitrogen (MUN)), somatic cells (e.g., in cells/ml), etc., expected to be present in milk flowing through inline sensor 102. As yet another example, inline sensor 102 can be configured to generate data that can be used to determine milk composition information indicative of an estimated amount and/or concentration (e.g., milligrams per deciliter (mg/dl), parts per million, percent by weight, percent by volume, etc.) of ancillary and/or trace components of milk such as concentration of somatic cells, blood, etc., expected to be present in milk flowing through inline sensor 102.

In some embodiments, inline sensor 102 can include any suitable hardware, firmware, and/or software that can be used to generate data that is suitable for determining a characteristic(s) of the milk flowing through inline sensor 102. For another example, inline sensor 102 can include hardware, firmware, and/or software that can be used to generate optical spectra data indicative of a presence and/or concentration of a particular molecule in the milk flowing through inline sensor 102. In a more particular example, inline sensor 102 can include a wavelength tunable light source (e.g., a swept-source laser, a frequency comb laser, etc.) configured to emit light of a particular wavelength or combination of wavelengths at a particular time toward milk flowing through a portion of inline sensor 102 (e.g., a light source that emits and/or can be controlled to emit a particular wavelength of light at a particular time that is known and/or can be detected, which can be a coherent light source such as a laser) and a detector (e.g., a photodetector or an array of photodetectors, such as a photodetector(s) configured to detect light in a wavelength range of the tunable light source) configured to detect light of a particular wavelength (e.g., based on a time at which the signal was sampled, and/or based on a wavelength of light directed toward the detector which samples the signal, such as via a grating) that has passed through, been reflected by, and/or been emitted by the milk.

As another example, inline sensor 102 can include hardware, firmware, and/or software that can be used to generate optical data indicative of a color(s) of light reflected and/or absorbed by the milk flowing through inline sensor 102. In a more particular example, inline sensor 102 can include a light source configured to emit light toward milk flowing through a portion of inline sensor 102 (e.g., a light source that emits light in multiple wavelengths, which can be a noncoherent light source) and an image sensor(s) (e.g., a color image sensor, such as a 1D or 2D CMOS or CCD sensor array(s) in which each pixel is associated with a particular color filter) configured detect an intensity of light of different colors that has passed through and/or been reflected by the milk passing through inline sensor 102 (e.g., many color image sensors use red, green, and blue filter arrays, but other combinations of filter colors, including some that include white and/or infrared pixels, can be used to characterize the intensity of light of different colors in light detected by an image sensor).

As yet another example, inline sensor 102 can include hardware, firmware, and/or software that can be used to generate electrical data indicative of one or more electric properties of milk flowing through inline sensor 102, such as a conductivity and/or any other suitable electric properties of the milk. In a more particular example, inline sensor 102 can include an electrode(s) configured to emit an electrical signal into milk flowing through a portion of inline sensor 102, and an electrode(s) configured to detect the electrical signal emitted into the milk flowing through the portion of inline sensor 102, and generate data indicative of an electrical property of the milk through which the signal was transmitted.

As still another example, inline sensor 102 can include hardware, firmware, and/or software that can be used to generate data indicative of a temperature of milk flowing through inline sensor 102.

As a further example, inline sensor 102 can include hardware, firmware, and/or software that can be used to generate data indicative of a volume of milk flowing through inline sensor 102.

In some embodiments, sensor 102 can include multiple different types of sensors in a single housing that can be used to generate data indicative of different characteristics of the milk flowing through inline sensor 102. For example, an inline sensor for determining characteristics of milk is described in Buciunas et al. International Patent Application Publication No. WO2024/075079, which can include a color sensor configured to generate color data that can be used to determine a characteristic(s) of milk that impacts color of milk (e.g., milk fat content, the presence of blood in the milk), a laser source and laser detector configured to generate spectral data that can be used to determine a characteristic(s) of milk that impacts how a particular wavelength(s) of light are absorbed and/or reflected by the milk (e.g., the presence and/or concentration of fat molecules, proteins, lactose, urea-containing molecules, etc.), and a contact sensor(s) configured to generate electric data that can that can be used to determine a characteristic(s) of milk (e.g., a concentration of somatic cells) that impacts an electrical property or properties (e.g., conductivity) of the milk, and/or configured to generate temperature data that can be used to more accurately determine a characteristic(s) of milk that is impacted by temperature (e.g., absorption of different wavelengths of light that can vary with concentration of a molecule(s) that absorbs the particular wavelength of light and the temperature of the milk, milk viscosity, temperature compensated conductivity, protein solubility, fat globule behavior, lactose crystallization, enzyme activity such as lactase vs alkaline phosphatase, milk pH, bacterial growth rates, etc.), and/or to monitor udder health (e.g., as inflammation and/or infection can cause increased udder temperature, leading to an increase in milk from the impacted area of the udder). In such an example, in addition to generating data suitable for determining a characteristic(s) of the milk flowing through inline sensor 102, inline sensor can include a sensor(s) that can generate data suitable for monitoring operation of a particular milking device (e.g., a milking device of milking station 106-1 providing milk to milk line 104-1 that is analyzed by inline sensor 102-1), such as data that can be used to determine a volume of milk being received from the particular milking device, a flow rate of milk being received from the particular milking device, etc.

As another example, inline sensors for determining characteristics of milk are marketed by, and available from, Brolis Sensor Technology (headquartered in Vilnius, Lithuania), which can be used to accurately determine at least fat content, protein content, and lactose content of milk flowing through the sensor.

In some embodiments, multiple inline sensors (e.g., in different housings) can be used to measure different characteristics of milk flowing through a milk line and/or to monitor operation of a milking device from which the milk is received. For example, a first inline sensor on a milk line (e.g., an inline sensor 102-1a on milk line 104-1 with a portion 100-1 of system 100 as shown in FIG. 1B) can be used to generate data that can be used to determine a first subset of characteristics of the milk flowing through the milk line, and one or more additional inline sensors (e.g., another sensor(s) and/or located at different points along milk line 104-1), such as a second inline sensor on the milk line (e.g., an inline sensor 102-1b on milk line 104-1 as shown in FIG. 1B) can be used to generate data that can be used to determine a second subset of characteristics of the milk flowing through the milk line. As a more particular example, first inline sensor 102-1a can be used to generate image data and spectral data that can be used to determine an amount and/or concentration of components of milk flowing through milk line 104-1 (e.g., main components, ancillary and/or trace components, contaminants, etc.), and second inline sensor 102-1b can be used to generate electric data and temperature data that can be used to determine a parameter(s) indicative of an amount of milk that is flowing through (and/or has flowed through) second inline sensor 102-1b (e.g., a volumetric flow, a total volume, etc.) and/or an amount and/or concentration of a component(s) of milk flowing through inline sensor 102-1b (e.g., a number and/or concentration of somatic cells). Note that while first inline sensor 102-1a is shown upstream of second inline sensor 102-1b, various sensors can be placed in any suitable order. In such an example, there may be overlap between characteristics that can be determined from data generated by first inline sensor 102-1a and second inline sensor 102-1b, such as both first inline sensor 102-1a and second inline sensor 102-1b generating data that can be used to determine a flow rate of milk, a temperature of the milk, and/or a conductivity of the milk, or there may be no overlap between the first subset of characteristics and second subset of characteristics.

In a yet more particular example, first inline sensor 102-1a can be an inline sensor described in Buciunas et al. International Patent Application Publication No. WO2024/075079 (e.g., which can be used to determine an amount and/or concentration of various milk components based on data from a color sensor, a laser detector, and contact sensor(s), and a flow rate of milk through the inline sensor based on data from the contact sensor(s)), and second inline sensor 102-1b can be an inline sensor described in Hanes et al. U.S. Pat. No. 10,598,528 (e.g., which can be used to determine at least a volumetric flow rate of milk through second inline sensor 102-1b based on data from contact sensor(s)).

In another yet more particular example, first inline sensor 102-1a can be a sensor from Brolis Sensor Technology, which can be used to determine at least fat content, protein content, lactose content, and temperature of the milk flowing through a milk line (e.g., milk line 104-1), and second inline sensor 102-1b can be a FloSmart sensor marketed by, and available from, BouMatic LLC (headquartered in Madison, Wisconsin, USA), which can be used to determine at least a volumetric flow of milk flowing through the milk line (e.g., milk line 104-1).

In some embodiments, inline sensor(s) 102 (and/or a particular type of inline sensor, if some milk lines are provided with multiple types of inline sensors) can be provided for a subset of N milk lines of M milking stations (and/or milk lines) in a dairy operation (e.g., dairy operation 120), and can be omitted from the other milk lines (e.g., the other M-N milk lines). For example, the subset of N milk lines which are provided with inline sensors 102 (e.g., inline sensors 102-1 to 102-N) can be a relatively small fraction of the M milking stations/milk lines (e.g., milking stations 106-1 to 106-M and/or milk lines 104-1 to 104-M), which can depend on a target fraction of animals from which at least some minimum number of samples are to be collected within a predetermined period of time. In such an example, the subset of N milk lines can be a fraction of M that is expected to generate milk characteristics for at least a predetermined fraction, F, of cows within a predetermined period of time T (e.g., corresponding to a week, two weeks, a month, half of an average lactation period for the cow, an entire lactation period, etc.). In a more particular example, if each cow is milked an average of two times per day, and a target is set to collect samples from at least 90% of the cows (e.g., there is a 10% or less chance that any individual cow is not milked at a milking station equipped with an inline sensor) during each seven day period, if N inline sensors are distributed among the M milking stations such that each cow has an equal chance (e.g., N/M) of being milked at a milking station associated with an inline sensor, the minimum value of N can be at least 15% of M (e.g., N can be chosen such that N≥0.15*M), which can be expected to generate milk characteristics for at least 90% of the cows at least once within any seven day period. As the number of samples per animal within a predetermined period of time increases (e.g., if identification of relatively short-term trends is desirable) and/or the target chance of collecting at least one sample during the time period increases, the minimum number N also increases. Additionally, as the predetermined period of time increases (e.g., from seven days to fourteen days), the minimum number N can be expected to decrease. In some embodiments, if a sensor can measure milk characteristic(s) with high accuracy, measurements during a single milking session may be sufficient to determine accurate milk quality metrics for a particular animal during the milking session. For example, an inline sensor that meets current standards promoted by the International Committee for Animal Recording (ICAR) is expected to measure fat, protein, and lactose content for each milking to an accuracy of within a range of ±0.1% to ±0.5%.

In some embodiments, any suitable milk quality metrics can be generated based on data collected by an inline sensor(s). For example, data collected using an inline sensor across multiple milking session can be used to determine metrics that characterize a milk quality characteristic over a particular period of time, such as average fat content, average protein content, trends in udder health over time, trends in milk urea nitrogen milk urea-nitrogen (MUN) over time, milk yield consistency, and fat to protein rations.

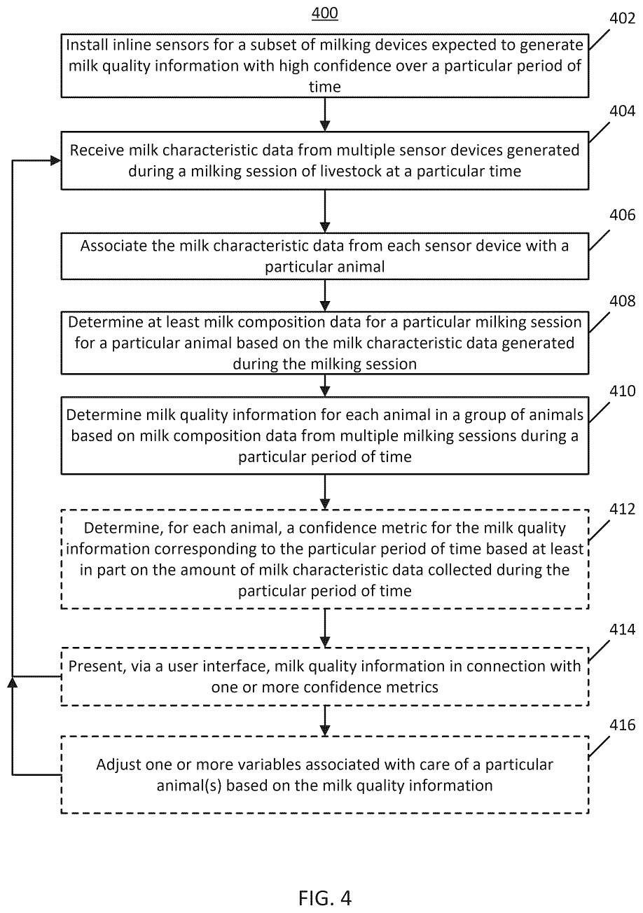

As described below in connection with FIG. 4, in some embodiments, milk characteristics from each cow can be monitored multiple times per month (e.g., as different cows are milked at different milking stations, some of which are provided with an inline sensor(s) and some which are not), and milk characteristic information for a particular cow over a predetermined period of time can be analyzed to calculate a confidence metric indicative of a reliability of the data, and/or determine that the data meets a reliability threshold (e.g., an ICAR standard). For example, milk characteristic information and an indication of reliability (e.g., an associated confidence metric, an indication that the milk characteristic information meets a predetermined reliability threshold) can inform a decision of a user of the milk characteristic information (e.g., an operator of the dairy, a veterinarian, etc.) of whether to adjust the care of the animal (e.g., adjusting an amount and/or composition of feed provided to the animal, adjusting whether and/or how often the animal is milked, whether to provide the animal with a particular treatment, etc.). As another example, milk characteristic information and an indication of reliability can inform an adjustment in automated system configured to provide care to the animal (e.g., an automated feed system, an automated milking system, etc.).

In some embodiments, milking stations 106 can be any suitable type of milking station. For example, milking stations 106 can be fully automated milking stations in which a robotic system prepares the cow for milking (e.g., including cleaning the teats) and attaches a milking device. As another example, milking stations 106 can be configured to facilitate machine milking in which a human operator prepares the cow for milking and attaches a milking device. Additionally, in some embodiments, milking stations 106 can be arranged in any suitable configuration. For example, milking stations 106 can be part of a rotary milking platform, a herringbone-style miking parlor, a tandem milking parlor, etc. Additionally or alternatively, milking stations 106 can include a mixture of different types of milking stations, and/or can be included in multiple different configurations.

In some embodiments, system 100 can include any suitable device or combination of devices that can be used to associate a particular animal with a milking station and/or inline sensor, which can be used to associate milk characteristic data and/or milk quality information with the animal which produced the milk. For example, system 100 can include one or more imaging devices can be used to capture image data of a milking station(s) and/or an animal associated with the milking station (e.g., as the animal is entering and/or exiting the milking station, as the animal is being milked at the milking station, etc.). As a more particular example, the image data can be analyzed to extract and/or read visually encoded data (e.g., alphanumeric text-based code such as an identification number printed on an ear tag or other surfaces, a machine-readable code such as a QR code affixed to the animal, etc.). As another more particular example, the image data can be analyzed to identify a particular animal based on the visual appearance of the animal in the image data (e.g., based on facial and/or other characteristics of the animal).

As another example, system 100 can include one or more wireless communication devices can be used to read information encoded in a device affixed to the animal. As a more particular example, one or more radio frequency identification (RFID) readers, near field communication (NFC) readers, ultrawideband (UWB) devices, Bluetooth devices, and/or any other suitable type of wireless communication technology that can be used for wireless identification, can be used to read identifying information encoded in a passive or active transmitter device affixed to the animal (e.g., as part of an ear tag, attached via a collar, implanted subcutaneously, etc.), and based on the location of the transmitter device, can associate a particular animal with a particular milking station. In such an example, the wireless communication device can be a fixed part of system 100, or a mobile device (e.g., a general purpose device such as a smartphone or wearable computing device, or a special purpose device configured to read data from a nearby transmitter affixed to an animal).

As yet another example, system 100 can receive input associating a particular animal (e.g., via a text-based code, an optical code, etc.) with a particular milking station. In a more particular example, such input can be received from a mobile device (e.g., a general purpose device such as a smartphone or wearable computing device executing a frontend of a milk quality analysis system, or a special purpose device configured to identify a milking station and an animal positioned at the milking station), or a fixed device (e.g., a keypad or touchscreen installed near a milking station). Additional particular examples of systems and devices that can be used to associate a particular animal with a milking station are described in Rajkondawar et al. U.S. Pat. No. 8,950,357, Siddell U.S. Pat. No. 8,955,459, and Hofman et al. U.S. Pat. No. 11,096,370, each of which is hereby incorporated by reference herein in its entirety.

In some embodiments, system 100 can include a livestock management system 112, which can be used to store information related to operation of dairy operation 120 and/or information related to livestock animals, such as cows milked at dairy operation 120. For example, livestock management system 112 can receive data from one or more sensors (e.g., inline sensors 102), devices, and/or systems (e.g., a system that associates a particular cow with a particular milking station), and/or input from one or more devices (e.g., input provided by an operator of dairy operation 120 via a user interface, such as a graphical user interface presented by a computing device). In a more particular example, livestock management system 112 can receive data indicative of one or more characteristics of milk flowed through an inline sensor (e.g., 102) during milking, and associate the data with the particular animal that produced the milk based on information indicating which animal was being milked at the milking station associated with the inline sensor during the time the data was generated.

Additionally, in some embodiments, livestock management system 112 can use received and/or stored data to determine any suitable information about a particular animal, which can be used to monitor and/or evaluate an animal. For example, livestock management system 112 can use received data to determine milk volume information, milk composition information, and/or any other suitable information that can be used in evaluating the production and/or health of a particular animal (e.g., a particular cow). In some embodiments, livestock management system 112 can be implemented locally and/or remotely. For example, livestock management system 112 can be implemented using only local devices within dairy operation 120 (e.g., sensors 102, local computing devices that receive and/or aggregate data from sensors 102, etc.). As another example, livestock management system 112 can be implemented using local devices within dairy operation 120 (e.g., sensors 102, local computing devices that receive and/or aggregate data from sensors 102, etc.) and remote devices (e.g., a remote server(s), a cloud computing service, etc.). As yet another example, livestock management system 112 can be implemented using primarily remote devices (e.g., a remote server(s), a cloud computing service, etc., that receives raw data from local sensor devices, such as inline sensors 102).

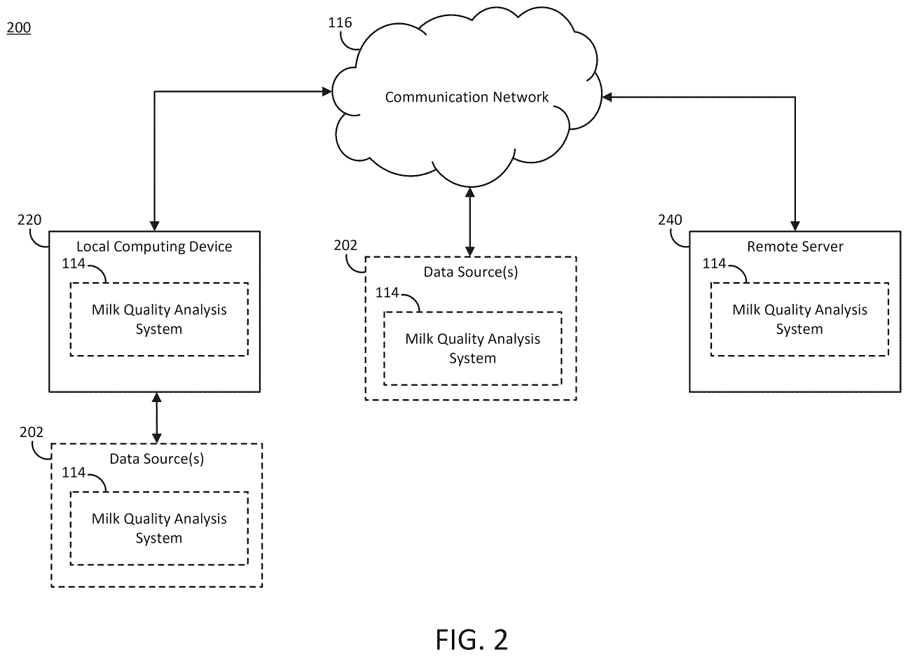

In some embodiments, system 100 can include a milk quality analysis system 114, which can be used to determine one or more milk quality metrics for each animal that has been milked at a milking station associated with a suitable inline sensor (e.g., milking station 106-1 and inline sensor 102-1) based on data generated by and/or received from the inline sensor. As described below, in some embodiments, milk quality analysis system 114 can be at least partially implemented in livestock management system 112 on a remote server (e.g., as a cloud application, within a software as a service platform, etc.). Additionally or alternatively, in some embodiments, milk quality analysis system 114 can be at least partially implemented in devices within dairy operation, such as sensors 102, a local computing device, etc. For example, as described below in connection with FIG. 2, milk quality analysis system 114 can be implemented as a distributed application across multiple devices, such as within a data source (e.g., inline sensor 102), within a local computing device (e.g., a mobile computing device such as a smartphone, tablet computer, etc., a laptop computer, a desktop computer, a local server, etc.) in communication with the data source, and/or within a remote computing device (e.g., a dedicated remote server, a device within a cloud computing system). In such an example, milk quality analysis system 114 can generate milk quality data and/or any other suitable data from raw sensor outputs (e.g., within inline sensor 102, at a local computing device, and/or at a remote computing device), aggregate milk quality data (e.g., within inline sensor 102, at a local computing device, and/or at a remote computing device), record milk quality data in connection with a particular animal (e.g., at a local computing device, and/or at a remote computing device), calculate additional milk quality data for a particular animal (e.g., over time) and/or for multiple animals (e.g., a particular herd) over any suitable period of time (e.g., at a local computing device, and/or at a remote computing device).

In some embodiments, data generated by a sensor(s) (e.g., inline sensors 102) can be transmitted to a computing device (e.g., a local computing device, a remote computing device, etc.) via a communication network 116, which can be any suitable communication network or combination of communication networks. For example, communication network 116 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard(s), such as CDMA, GSM, LTE, LTE Advanced, 5G NR, etc.), a wired network, etc. In some embodiments, communication network 116 can include one or more portions of a local area network (LAN), a wide area network (WAN), a public network (e.g., the Internet, which may be part of a WAN and/or LAN), any other suitable type of network, or any suitable combination of networks. Communications links shown in FIGS. 1 to 3 can each be any suitable communications link or combination of communications links, such as wired links, fiber optic links, Wi-Fi links, Bluetooth links, cellular links, etc.

In some embodiments, system 100 can include farm automation equipment 118, which can include any suitable devices configured to automate one or more tasks associated with dairy operation 120 and/or collect data related to dairy operation 120 and/or animals associated with dairy operation 120. For example, farm automation equipment 118 can include an automated feeding system (e.g., a robotic feeding system) that uses one or more devices to automatically dispense feed (e.g., a particular quantity and/or composition of feed) to be consumed by one or more particular animals at least partially without human intervention. In such an example, the amount and/or composition of the feed can be based on data stored in livestock management system, such as data related to health information, milk quality information, and/or milk production information associated with the animal for which the feed is being dispensed. As another example, farm automation equipment 118 can include an automated milking system (e.g., a robotic milking system) that uses one or more devices to automatically milk cows at least partially without human intervention.

In some embodiments, farm automation equipment 118 can be controlled to operate on a predetermined schedule, in response to sensor data (e.g., in response to determining that a particular condition has been satisfied, such as that a particular animal has entered a feeding area) measured by and/or received by farm automation equipment 118, and/or based on instructions from a computing device (e.g., a computing device implementing livestock management system 112). For example, in some embodiments, farm automation equipment 118 can receive instructions from livestock management system 112 to dispense a particular amount of one or more types of feed and/or supplements (e.g., to treat and/or prevent one or more conditions that adversely impact the health and/or milk production of the cow) to a particular cow, and can autonomously dispense the instructed type(s) feed and/or supplements in the instructed amount to the cow based on a location of the cow.

In some embodiments, farm automation equipment 118 can be used to monitor certain animal activity (e.g., in addition to, or in lieu of, activity monitoring sensor(s) 122) using any suitable sensor(s). For example, farm automation equipment 118 can monitor feed consumption for each animal using any suitable sensor(s) and/or combination of sensors. In a more particular example, farm automation equipment 118 can use one or more imaging devices (e.g., for capturing digital images, video, 3D scene depth information, etc.) to determine how much feed has been consumed by a particular animal. In a yet more particular example, farm automation equipment 118 can use one or more computer vision techniques (e.g., which may or may not incorporate machine learning techniques) to estimate an amount of feed dispensed to the animal (which may also be determined based on an amount of feed that a robotic feeding system was instructed to dispense to the animal and/or an amount of feed that a robotic feeding system reported dispensing to the animal), and/or to estimate an amount of feed consumed by the animal (e.g., based on an estimate of how much feed is remaining and an amount of feed that was dispensed by the animal). In another more particular example, farm automation equipment 118 can use one or weighing devices (e.g., a scale) to determine how much feed has been consumed by a particular animal (e.g., based on a weight of feed that was provided and a weight of feed that remained when the animal finished feeding).

FIG. 2 shows an example of a system 200 for analyzing milk quality using data from inline sensors in accordance with some embodiments of the disclosed subject matter. As shown in FIG. 2, a local computing device 220 can receive data from a data source 202. In some embodiments, local computing device 220 can execute at least a portion of milk quality analysis system 114 to determine milk quality information based on data generated by, and/or received from, data source 202. Additionally or alternatively, in some embodiments, local computing device 220 can communicate data received from data source 202 to a server 240 over a communication network 116 and/or server 240 can receive data from data source 202 (e.g., directly and/or indirectly using communication network 116), which can execute at least a portion of milk quality analysis system 114. In such embodiments, server 240 can return information to local computing device 220 (and/or any other suitable computing device), such as milk quality information stored by and/or determined by milk quality analysis system 114. In some embodiments, milk quality analysis system 114 can execute one or more portions of process 400 described below in connection with FIG. 4, and/or any other suitable processes.

In some embodiments, local computing device 220 and/or server 240 can be any suitable computing device or combination of devices, such as a desktop computer, a laptop computer, a smartphone, a tablet computer, a wearable computer, a server computer, a virtual machine being executed by a physical computing device, etc.

In some embodiments, data source(s) 202 can be any suitable source(s) of data that can be used to determine milk quality information for a particular dairy livestock (e.g., a cow) or a group of dairy livestock (e.g., a herd or a portion of a herd) as described herein. For example, data source(s) 202 can include an inline sensor(s) (e.g., one or more of inline sensors 102 described above in connection with FIGS. 1A and 1B). As another example, data source(s) 202 can include a device(s) that is configured to measures and/or determine a position, posture, activity, etc., of one or more animals. In a more particular example, data source(s) 202 can include devices described in Rajkondawar et al. U.S. Pat. No. 8,949,034, which is hereby incorporated by reference herein in its entirety, that are configured to measure and/or determine that position, posture, and activity of cows in a monitored area. As another more particular example, data source(s) 202 can include devices affixed to particular cows that are configured to that are configured to measure and/or determine the position, posture, and activity of cows (e.g., a monitoring device affixed with a collar).

In some embodiments, one or more data sources 202 can be in direct communication with local computing device 220 (e.g., local computing device 220 can be located within the same building as inline sensors 102). For example, data source 202 can communicate with local computing device 220 via a direct wired connection or a direct wireless connection (e.g., a peer-to-peer connection). Additionally or alternatively, in some embodiments, one or more data sources 202 can be located locally to, and/or remotely from, local computing device 220, and can communicate data (e.g., data generated by an inline sensor or other data source) and/or information (e.g., information calculated by an inline sensor or other data source) to local computing device 220 (and/or server 240) via a communication network (e.g., communication network 116).

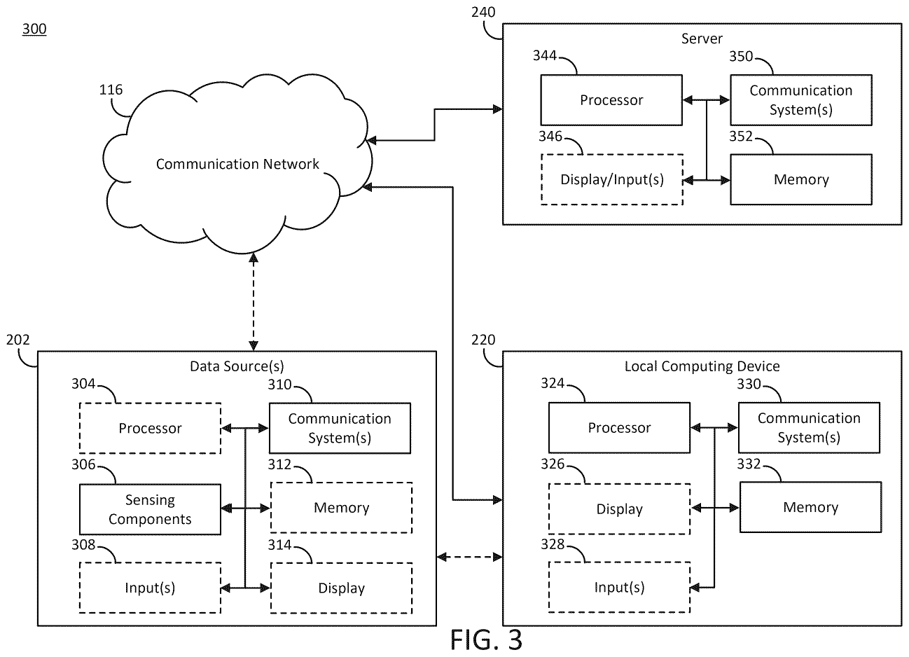

FIG. 3 shows an example of hardware 300 that can be used to implement data source 202, computing device 220, and server 240 of FIG. 2 in accordance with some embodiments of the disclosed subject matter. As shown in FIG. 3, in some embodiments, data source(s) 202 can include a processor 304, sensing components 306, one or more inputs 308, one or more communication systems 310, memory 312, and/or a display(s) 314. In some embodiments, processor 304 can be any suitable hardware processor or combination of processors, such as a central processing unit (CPU), an accelerated processing unit (APU), a graphics processing unit (GPU), a field programmable gate array (FPGA), an application specific integrated circuit (ASIC), a microcontroller, etc. In some embodiments, sensing components 306 can include components that can be used to generate data that can be used to monitor milk quality of dairy livestock, such as one or more optical components, one or more contact sensors, etc. that can be used to measure properties of milk passing through an inline sensor. Additionally, in some embodiments, sensing components 306 can include components that can be used to generate data that can be used to monitor the position, posture, activity, etc., of dairy livestock, such as one or more electrical sensors (e.g., an RFID reader, a NFC reader, an UWB transceiver, a Bluetooth transceiver, a global navigation satellite system (GNSS) receiver such as a global position system (GPS receiver), etc.), one or more imaging devices (e.g., one or more digital cameras, thermal cameras, etc.), one or more inertial measurement devices (e.g., one or more accelerometers, gyroscopes, etc.). In some embodiments, inputs 308 can include any suitable input devices and/or sensors that can be used to receive user input, such as a keyboard, a mouse, a touchscreen, a touchpad, a microphone, a camera, etc. In some embodiments, display 314 can include any suitable display devices, such as a touchscreen, a computer monitor, a television, etc. In some embodiments, data source(s) 202 can omit processor 304 and/or memory 312 (e.g., where data source 202 is an inline sensor configured to stream raw data to local computing device 220 device or server 204). Additionally, in some embodiments, data source(s) 202 can omit inputs 308, and/or a display(s) 314 (e.g.,, where data source 202 that is not configured for direct user interaction). For example, data source 202 can provide data, results of an analysis, etc., to local computing device 220 and/or server 240, and local computing device 220 and/or server 240 can use the data, results, and/or a user interface to execute at least a portion of a process(es) described herein, present milk quality information, present a user interface, etc.

In some embodiments, sensing components 306 can include components that are used to measure data indicative of milk quality and/or animal health. For example, in some embodiments, data source 202 can include sensing components used to implement an inline sensor(s) (e.g., one or more of inline sensors 102 described above in connection with FIGS. 1A and 1B). As another example, in some embodiments, data source 202 can include sensing components used to implement an activity monitoring sensor(s). As yet another example, in some embodiments, data source 202 can include sensing components used to implement a mobile computing device, such as an internet-of-things (IoT) device, a smartphone, a tablet computer, a laptop computer, a wearable computing device, etc.

In some embodiments, communication system(s) 310 can include any suitable hardware, firmware, and/or software for communicating information over a communication network 116 and/or any other suitable communication networks. For example, communication systems 310 can include one or more transceivers, one or more communication chips and/or chip sets, etc., that can be used to establish a wired and/or wireless communication link. In a more particular example, communication systems 310 can include hardware, firmware, and/or software that can be used to establish a direct or indirect wired connection and/or a direct or indirect wireless connection, such as a Bluetooth connection, Bluetooth Low Energy connection, an UWB connection, an NFC connection, a ZigBee connection, a Wi-Fi connection, a cellular connection (e.g., an uplink connection, a downlink connection, or a sidelink connection), an Ethernet connection, etc.

In some embodiments, memory 312 can include any suitable storage device or devices that can be used to store instructions, values, etc., that can be used, for example, by processor 304 to perform processes described herein, to capture data, to store data, to retrieve data, etc., to communicate with local computing device 220 and/or server 240 via communication system(s) 310, etc. Memory 312 can include any suitable volatile memory, non-volatile memory, storage, or any suitable combination thereof. For example, memory 312 can include random access memory (RAM), read-only memory (ROM), electronically erasable programmable read-only memory (EEPROM), one or more flash drives, one or more hard disks, one or more solid state drives, one or more optical drives, etc.

In some embodiments, memory 312 can have encoded thereon a computer program for controlling operation of data source 202. In such embodiments, processor 304 can execute at least a portion of the computer program to: generate raw measurement data, determine milk characteristic data (e.g., based on the raw data), generate one or more milk quality metrics, store milk characteristic data and/or milk quality metrics in memory 312, retrieve milk characteristic data and/or milk quality metrics from storage in memory 312, transmit information (e.g., milk characteristic data and/or milk quality metrics) to local computing device 220 and/or server 240, to execute at least a portion of a process for monitoring milk quality, such as one or more portions of processes described below in connection with FIG. 4, etc.

In some embodiments, local computing device 220 can include a processor 324, a display 326, one or more inputs 328, one or more communication systems 330, and/or memory 332. In some embodiments, processor 324 can be any suitable hardware processor or combination of processors, such as a CPU, an APU, a GPU, an FPGA, an ASIC, a microcontroller, etc. In some embodiments, display 326 can include any suitable display devices, such as a computer monitor, a touchscreen, a television, etc. In some embodiments, inputs 328 can include any suitable input devices and/or sensors that can be used to receive user input, such as a keyboard, a mouse, a touchscreen, a microphone, etc. In some embodiments, local computing device 220 can omit inputs (e.g., where local computing device 220 is a backend device that is not configured for direct user interaction). For example, local computing device 220 can provide results of an analysis, raw measurement data, milk characteristic data and/or milk quality metrics, and/or a portion of a user interface to another computing device (e.g., a frontend device configured for direct user interaction), which can use the data, metrics, etc., and/or user interface to execute at least a portion of a process(es) described herein, present data and/or information, present a user interface, etc. In some embodiments, any suitable computing device (e.g., a desktop computer, a server computer, a laptop computer, a smartphone, a tablet computer, a wearable computer, a virtual machine being executed by a physical computing device, etc.) can be used to implement local computing device 220.

In some embodiments, communication systems 330 can include any suitable hardware, firmware, and/or software for communicating information over communication network 116 and/or any other suitable communication networks. For example, communication systems 330 can include one or more transceivers, one or more communication chips and/or chip sets, etc., that can be used to establish a wired and/or wireless communication link. In a more particular example, communication systems 330 can include hardware, firmware, and/or software that can be used to establish a direct or indirect wired connection and/or a direct or indirect wireless connection, such as a Bluetooth connection, a Bluetooth Low Energy connection, an UWB connection, an NFC connection, a ZigBee connection, a Wi-Fi connection, a cellular connection (e.g., an uplink connection, a downlink connection, or a sidelink connection), an Ethernet connection, etc.

In some embodiments, memory 332 can include any suitable storage device or devices that can be used to store instructions, values, etc., that can be used, for example, by processor 324 to communicate with data source 202 and/or server 240 via communication system(s) 330, receive data from data source device(s) 202, receive additional data and/or instructions via inputs 328 and/or from a remote computing device (e.g., server 240), determine milk characteristic data and/or milk quality metrics, analyze milk characteristic data and/or milk quality metrics associated with a dairy livestock or group of dairy livestock, provide milk characteristic data and/or milk quality metrics to a remote computing device for analysis (e.g., server 240), to present a user interface that includes milk characteristic data and/or milk quality metrics, etc. Memory 332 can include any suitable volatile memory, non-volatile memory, storage, or any suitable combination thereof. For example, memory 332 can include RAM, ROM, EEPROM, one or more flash drives, one or more hard disks, one or more solid state drives, one or more optical drives, etc.

In some embodiments, memory 332 can have encoded thereon a computer program for controlling operation of local computing device 220. In such embodiments, processor 324 can use the computer program to: receive raw measurement data (e.g., from data source(s) 202), determine milk characteristic data (e.g., based on the raw data), receive milk characteristic data (e.g., from data source(s) 202), generate one or more milk quality metrics (e.g., based on milk characteristic data), store milk characteristic data and/or milk quality metrics in memory 332, retrieve milk characteristic data and/or milk quality metrics from storage in memory 332, transmit information (e.g., milk characteristic data and/or milk quality metrics) to another local computing device and/or server 240, to execute at least a portion of a process for monitoring milk quality, such as one or more portions of processes described below in connection with FIG. 4, etc.

In some embodiments, server 240 can include a processor 344, a display and/or input(s) 346, a communication system(s) 350, and/or memory 352. In some embodiments, processor 344 can be any suitable hardware processor or combination of processors, such as a CPU, an APU, a GPU, an FPGA, an ASIC, a microcontroller, etc.

In some embodiments, display and/or input(s) 346 can include any suitable display devices, such as a computer monitor, a touchscreen, a television, etc., and/or can include any suitable input devices and/or sensors that can be used to receive user input, such as a keyboard, a mouse, a touchscreen, a microphone, etc. In some embodiments, server 240 can omit display/input(s) 346 (e.g., where server 240 is not configured for direct user interaction). For example, server 240 can communicate with another computing device (e.g., local computing device 220, data source 202, etc.) via communication system(s) 350 to: receive raw measurement data (e.g., from data source(s) 202), receive milk characteristic data (e.g., from data source(s) 202 and/or local computing device 220), transmit information (e.g., milk characteristic data and/or milk quality metrics) to local computing device 220, another server, and/or a different user computer device (e.g., which may or may not be located in or near dairy operation 120), provide data and/or a portion of a user interface to be used to present milk quality information, to present a user interface that includes milk quality information, etc.

In some embodiments, communication systems 350 can include any suitable hardware, firmware, and/or software for communicating information over communication network 116 and/or any other suitable communication networks. For example, communication systems 350 can include one or more transceivers, one or more communication chips and/or chip sets, etc., that can be used to establish a wired and/or wireless communication link. In a more particular example, communication systems 350 can include hardware, firmware, and/or software that can be used to establish a direct or indirect wired connection and/or a direct or indirect wireless connection, such as a CAN bus connection, a Bluetooth connection, Bluetooth Low Energy connection, an UWB connection, an NFC connection, a ZigBee connection, a Wi-Fi connection, a cellular connection (e.g., an uplink connection, a downlink connection, or a sidelink connection), an Ethernet connection, etc.

In some embodiments, memory 352 can include any suitable storage device or devices that can be used to store instructions, values, etc., that can be used, for example, by processor 344 to communicate with data source 202, local computing device 220, etc., via communication system(s) 350, etc. Memory 352 can include any suitable volatile memory, non-volatile memory, storage, or any suitable combination thereof. For example, memory 352 can include RAM, ROM, EEPROM, one or more flash drives, one or more hard disks, one or more solid state drives, one or more optical drives, etc.

In some embodiments, memory 352 can have encoded thereon a computer program for controlling operation of server 240. In such embodiments, processor 344 can use the computer program to: receive raw measurement data (e.g., from data source(s) 202), determine milk characteristic data (e.g., based on the raw data), receive milk characteristic data (e.g., from data source(s) 202 and/or local computing device 220), generate one or more milk quality metrics (e.g., based on milk characteristic data), store milk characteristic data and/or milk quality metrics in memory 352, retrieve milk characteristic data and/or milk quality metrics from storage in memory 352, transmit information (e.g., milk characteristic data and/or milk quality metrics) to local computing device 220, another server, and/or a user computing device (e.g., a smartphone, tablet computer, laptop computer, desktop computer, wearable computer, etc.), to execute at least a portion of a process for monitoring milk quality, such as one or more portions of processes described below in connection with FIG. 4, etc.

FIG. 4 shows an example of a process 400 for automated milk quality monitoring using inline sensors in accordance with some embodiments of the disclosed subject matter.

At 402, process 400 can include installing inline sensors (e.g., an inline sensor(s) described above in connection with FIGS. 1A and 1B) for a subset of milking devices that is expected to generate milk quality information with a high confidence over a particular period of time. In some embodiments, inline sensors installed at 402 can be incorporated into milking equipment (e.g., can be integrated into a milk line), or can be separate from milking equipment that has already been installed and/or is being installed. For example, inline sensors installed at 402 can be added to an existing milk line.

In some embodiments, the number of inline sensors installed at 402 can be at least a minimum number such reliable (e.g., high confidence) information about the milk quality of all dairy livestock (e.g., cows) milked at a particular milking operation over a predetermined period of time. As described above, as the predetermined time period decreases, the ratio of inline sensors to milking stations may need to be increased to generate reliable data for all cows over the predetermined time period that can be used to inform decisions for each cow. For example, a relatively small fraction of milk lines (e.g., about 10%) can be equipped with inline sensors to generate reliable milk quality information for each cow in a herd over longer periods of time (e.g., multiple weeks, a month, or more). As the period of time over which reliable data is desired increases, the fraction of milk lines that need to be equipped with inline sensors generally decreases. In such an example, milk quality data for each cow can be used to inform decisions about breeding selection for increasing desirable traits and/or avoiding undesirable traits. As a more particular example, to reliably collect samples from at least 90% of cows in a herd every week when milking twice per day, about 15% of milking stations can be equipped with inline sensors. As another example, a larger fraction of milk lines (e.g., about 30% or more) can be equipped with inline sensors to generate reliable milk quality information for each cow in a herd over a shorter period of time (e.g., less than a week or multiple times per week). In such an example, milk quality data for each cow can be used to monitor health of cows that are being milked, and inform actions to mitigate health conditions that are difficult to detect and treat economically using conventional laboratory testing and/or routine veterinary care. As a more particular example, to reliably collect samples from at least 90% of cows in a herd every three to four days when milking about twice per day, about 30% of milking stations can be equipped with inline sensors. As another more particular example, to reliably collect two samples from at least 90% of cows in a herd twice per week when milking about twice per day, about 40% of milking stations can be equipped with inline sensors. Note that sensors may be installed in groups of predetermined size, and the number of milk lines being monitored may exceed the minimum number. For example, if inline sensors are installed in sets of eight, a milking parlor with 40 to 50 milking stations can be equipped with inline sensors (e.g., inline sensors 102) on eight milk lines if the target fraction of lines is 15%, sixteen (two sets of eight) milk lines if the target fraction of lines is 30%, and sixteen or twenty four (three sets of eight) milk lines if the target fraction of lines is 40% (e.g., depending on the total number of milking stations). In some embodiments, incidence of a metabolic condition, such as NEB, in a herd can be estimated based on testing of a statistically significant sample of animals (e.g., at least twelve animals at once).

At 404, process 400 can receive milk characteristic data from multiple inline sensor devices that each generated the milk characteristic data during a milking session of livestock at a particular time. Additionally or alternatively, in some embodiments, at 404, process 400 can receive raw sensor outputs from multiple inline sensor devices, and calculate milk characteristic data from the raw sensor outputs.

In some embodiments, milk characteristic data and/or raw outputs can be received periodically (e.g., at regular and/or irregular intervals) during a milking session as values are generated. For example, the inline sensors can transmit milk characteristic data and/or raw outputs as the data and/or raw outputs are generated during a milking session. As another example, the inline sensors can transmit milk characteristic data and/or raw outputs at predetermined intervals. Additionally or alternatively, in some embodiments, milk characteristic data and/or raw outputs can be received when a milking session is complete. For example, the inline sensors can transmit milk characteristic data and/or raw outputs for an entire milking session after a milking device has completed milking a particular cow. In some embodiments, process 400 can receive milk characteristic data and/or raw outputs from multiple inline sensor devices concurrently (e.g., data from two inline sensors generated at about the same time can be received at about the same time).

In some embodiments, timing information can be associated with received milk characteristic data and/or raw outputs from an inline sensor. For example, process 400 can associate a time at which milk characteristic data and/or raw outputs was received with the data. As another example, milk characteristic data and/or raw outputs can be associated with a time stamp (e.g., generated by the inline sensor).

In some embodiments, identifying information of an inline sensor that generated milk characteristic data and/or raw outputs can be associated with the received milk characteristic data and/or raw outputs from an inline sensor. For example, different inline sensors can transmit information using different communication channels (e.g., different wired or wireless connections, different ports, different destination addresses, etc.), which can be used to identify a source of data received via a particular communication channel. As another example, different inline sensors can be assigned different address information (e.g., a unique IP address, a unique MAC address, etc.), which can be used to identify a source of data received from a data source.

At 406, process 400 can associate the received milk characteristic data from each sensor device with a particular animal. For example, in some embodiments, process 400 can associate data received from a particular sensor with a particular animal based on which animal was being milked at the milking station associated with the particular sensor at the time when the data was generated by the sensor. In such an example, process 400 can receive information indicating which animal is being milked at a particular milking station, and/or which milking station is being used to milk a particular animal, from any suitable device or system (e.g., devices and/or systems described above in connection with FIG. 1A).

At 408, process 400 can determine at least milk composition data for a particular milking session for a particular animal based on the milk characteristic data for the particular milking session. In some embodiments, process 400 can use milk characteristic data to determine milk composition data using any suitable technique(s). For example, if milk characteristic data is milk composition data of milk flowing through an inline sensor at a particular time, process 400 can use data about the volume of milk flowing through the inline sensor a time when the milk characteristic data was generated to determine milk composition data for a period of time.

In some embodiments, process 400 can determine any suitable milk composition data at 408, such as one or more of fat content, protein content, lactose content, etc. Additionally, in some embodiments, process 400 can determine any other suitable values indicative of milk quality based on the received milk characteristic data, such as whether and/or in what concentration one or more contaminants, metabolites, analytes, etc., are present, a volume of milk produced during the session, etc.

At 410, process 400 can determine milk quality information for each animal in a group of animals based on milk composition data from multiple milking sessions during a particular period of time. In some embodiments, the milk quality information can be based on milk composition data from any suitable number of milking sessions and/or over any suitable period of time. In some embodiments, the milk quality information can include one or more milk quality metrics, such as fat content for each milking session, a value indicative of how the fat content for each milking session compares to average fat content across any suitable population (e.g., all cows, other cows in the herd, etc.), a value indicative of how the fat content for each milking session compares to average fat content of milk produced by the particular cow, average fat content for a set of milking sessions, variance in fat content across milking sessions, etc.

At 412, process 400 can determine, for each animal, a confidence metric for the milk quality information corresponding to the particular period of time based at least in part on the amount of milk characteristic data that was collected during the particular period of time. In some embodiments, process 400 can use any suitable technique(s) to determine a confidence metric for milk quality information. In some embodiments, the confidence metric can be expressed using any suitable technique or combination of techniques (e.g., as a percent confidence, a margin of error, error bars, etc.). In some embodiments, process 400 can omit 412, for example, where the system and/or devices being used to execute process 400 (e.g., inline sensors 102, milk quality analysis system 114) has been certified as complying with a particular standard (e.g., one or more ICAR standards). In such an example, if the amount of data that has been collected for a particular cow is insufficient to determine a milk quality metric for a particular period of time, process 400 can indicate that the milk quality information does not comply with the standard and/or can inhibit the data from being presented for that period of time.

At 414, process 400 can present, via a user interface, milk quality information. In some embodiments, process 400 can present the milk quality information in response to a request from a user computing device (e.g., in response to a request from a web browser or dedicated application being executed by the user computing device). Additionally or alternatively, in some embodiments, process 400 can cause the milk quality information to be presented to a user in response to one or more conditions being satisfied (e.g., a particular milk quality metric associated with a particular cow being above or below a threshold, or outside of a particular range, a determination that a particular cow is in need of attention based on the milk quality information, etc.). In some embodiments, the user interface can include one or more confidence metrics and/or other indications that the data is sufficiently reliable (e.g., an indication that the data was generated to comply with ICAR standards).

In some embodiments, the information presented at 414 can be used to inform decisions by a user (e.g., a farmer, a breeder, a veterinarian, etc.) about the care and/or management of the cow(s) associated with the information. For example, the information presented at 414 can inform a decision about whether to provide a particular cow with more or less feed and/or with a different composition of feed. As another example, the information presented at 414 can inform a determination of whether a particular cow is likely to be impacted by a particular health condition(s).

In some embodiments, the information presented at 414 can be presented within a user interface that can be used to provide input to adjust one or more variables associated with care of a particular animal(s). For example, a user (e.g., a farmer, a breeder, a veterinarian, etc.) can make a decision about the care and/or management of the cow(s) associated with information presented in the user interface, and can provide input to adjust one or more variables associated with care of a particular animal(s). In such an example, the input can cause automated equipment (e.g., automated farm equipment 118) to adjust operation in connection with the cow(s) for which the input was provided.

In some embodiments, process 400 can omit 414, for example where a system executing process 400 (e.g., livestock management system 112) is configured to autonomously adjust one or more variables associated with care of a particular animal(s) based on the milk quality information without user intervention. Note that, in some embodiments, the milk quality information and/or other livestock management information can be accessible and/or presented to a user via a user interface via such a system, but presentation of such information may not be required for the system to make adjustments to the care of a particular animal(s). Additionally, in some embodiments, a user can be provide input (e.g., via a user interface) to make adjustments to the care of a particular animal(s), but such input may not be required for the system to make adjustments to the care of a particular animal(s).

At 416, process 400 can adjust (e.g., in response to user intervention, or without user intervention) one or more variables associated with care of a particular animal(s) based on the milk quality information. In some embodiments, process 400 can use any suitable technique or combination of techniques to determine whether to, and how to, adjust a variable(s) based on the milk quality information. For example, process 400 can determine whether to adjust an amount and/or composition of feed being provided to a particular cow based on the yield, the fat content, and/or protein content of the milk from a milking or multiple milkings. As a more particular example, if the milk quality data indicates that a cow's milk is low in fat or protein (e.g., compared to average fat and/or protein content across a group of cows that includes the compared to an desired or expected fat or protein content for that cow, compared to the historic average for the cow, etc.), the amount of feed that the cow receives can be adjusted to ensure that the cow is getting sufficient nutrients to boost milk production and/or improve milk composition. As another more particular example, if milk urea nitrogen (MUN) levels are high, it can indicate that the cow is not efficiently utilizing protein, and the composition of the feed can be adjusted (e.g., by adding more fiber, protein, or energy-dense foods) to help the cow more efficiently utilize the nutrients, which can be expected to produce higher-quality milk.

As another example, process 400 can determine whether to adjust how often a cow is milked. In a more particular example, if milk quality data includes a dip in milk quality and/or yield, the milking frequency can be adjusted to increase how often the cow is milked (e.g., increasing the number of milkings per day, decreasing the interval between milkings, etc.), which can help increase milk production and/or relieve pressure on the udder. As another more particular example, if milk quality data (e.g., elevated temperature) is indicative of mastitis and/or high somatic cell counts (which can indicate infection), the milking frequency can be adjusted to decrease how often the cow is milked and/or give the cow more time to rest.

As yet another example, process 400 can determine whether to further evaluate the health of the cow and/or treat the cow for a health condition determined from the milk quality data. In a more particular example, when milk quality starts to decline, it can be a signal that the health of the cow is in decline (e.g., if other sources of decline are not evident from the data), such as if the milk has elevated MUN and/or if the milk temperature is elevated (e.g., relative to a baseline(s), such as a temperature of milk from other cows milked during the same time period), it can indicate that the cow is experiencing mastitis and/or other health issues, and a user can be alerted to the potential declining health of the cow (e.g., the information can be provided to an operator at the dairy and/or a veterinarian, and the user and/or veterinarian can more closely evaluate and/or monitor the health of the cow, begin treating an identified condition, change the routine of the cow, etc., to improve the health of the cow). In another more particular example, if the composition of the milk produced by the cow indicates that the cow is experiencing a nutritional deficiency (e.g., a negative energy balance), a supplement (e.g., a high energy component of fed, such as propylene glycol) can be added to the cow's feed to address the nutritional deficiency.

As still another example, process 400 can determine whether changes in environmental conditions may be causing milk quality issues, such as if external factors, such as temperature, humidity, or stress are likely to impact the milk quality and/or yield of a cow (e.g., if cows are uncomfortable, it can affect how the cows produce milk). In such an example, if milk quality has declined (and/or if measured environmental conditions have changed), it can indicate that environmental conditions are sub-optimal (e.g., in addition to one or more other potential causes of decline in milk quality, and/or as a primary cause of decline in milk quality (e.g., if other sources of decline are not evident from the data). In such an example, adjustments can be made to improve barn conditions, such as setting up or activating a cooling system(s), changing how cows are housed, etc., to help cows feel more comfortable, which can lead to improvements in milk quality.

As a further example, process 400 can determine whether to suggest or consider a particular breeding decision for a cow based on milk quality over time. In a more particular example, if the quality of milk produced by a cow is generally poor (e.g., there are persistent and/or recurring indications of health and/or metabolic problems over time that are not economically resolved with treatment), a user can deprioritize breeding of that cow (e.g., by focusing breeding on cows that consistently produce higher-quality milk and/or that do not have persistent and/or recurring health and/or metabolic problems, by culling the cow, etc.). As another more particular example, if the quality of milk produced by a cow is generally high (e.g., the cow consistently produces high quality milk, the cow does not have persistent and/or recurring health and/or metabolic problems, the cow quickly recovers from health and/or metabolic problems, etc.), a user can deprioritize breeding of that cow.

In some embodiments, process 400 can adjust one or more variables associated with care of a particular animal(s) to effect a treatment of prophylaxis for a particular disease or medical condition of the animal. For example, if process 400 determines that the animal is experiencing a negative energy balance, process 400 can, at 416, cause glycol to be added to the animals feed to treat the negative energy balance, and/or to prevent the negative energy balance from progressing to ketosis.

In some embodiments, process 400 can determine whether to adjust one or more variables associated with care of the particular animal(s) based a confidence in a determination that adjusting the variable is called for (e.g., a likelihood that it will lead to an improvement in the health of the animal, a likelihood that the health of the animal will deteriorate without the adjustment, a likelihood that the adjustment leads to an increase in production by the animal, etc.). Additionally or alternatively, in some embodiment, process 400 can prompt a user (e.g., a farmer, a veterinarian, etc.) to indicate whether the adjustment is authorized an adjustment prior to implementing the adjustment. For example, if a condition or set of conditions is satisfied (or not satisfied), process 400 can determine that authorization is required before implementing an adjustment identified based on the milk quality information. In a more particular example, if confidence for the adjustment is above a threshold, process 400 can determine that authorization is not required, but if the confidence for the adjustment is below the threshold but above a secondary threshold, process 400 can determine that authorization is required. As another more particular example, if the adjustment is likely to cost over a threshold amount, process 400 can determine that authorization is required. As yet another more particular example, if the adjustment is to one or more predetermined variables (e.g., related to treatment of certain health conditions), process 400 can determine that authorization is required.

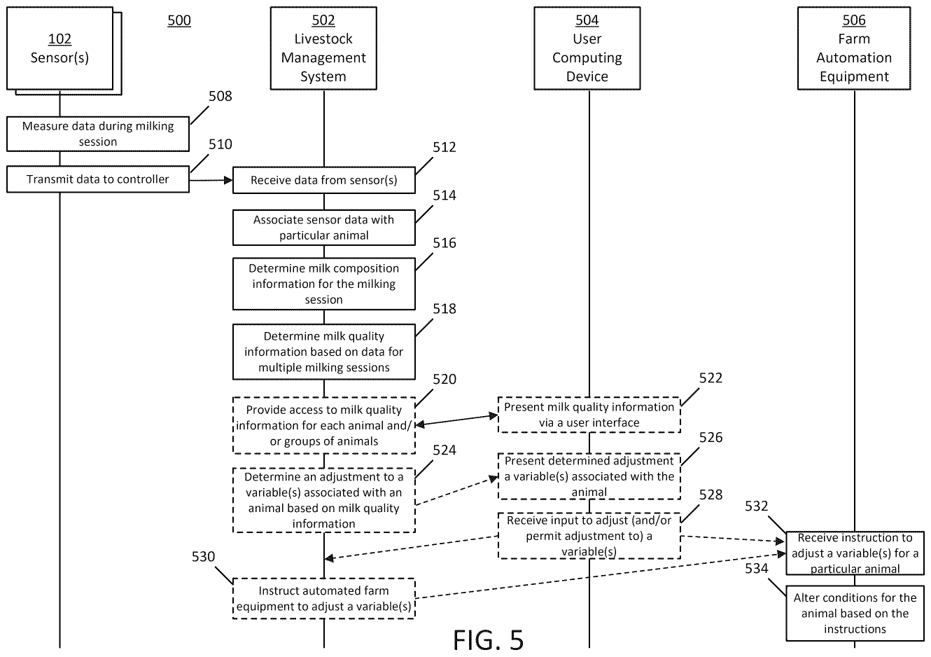

FIG. 5 shows an example flow 500 for automated milk quality monitoring using inline sensors in accordance with some embodiments of the disclosed subject matter. In FIG. 5, example flow 500 can include various devices, such as sensors 102 (e.g., one or more of inline sensors 102 described above in connection with FIGS. 1A and 1B, installed on a subset of milk lines), a livestock management system 502 (e.g., including one or more of processors 324 and 344), a user computing device 504 (e.g., a smartphone, a tablet computer, a laptop computer, a desktop computer, a wearable computing device, etc.), and farm automation equipment 506 (e.g., as described above in connection with FIGS. 1A, 1B, and 4).

At 508, sensors 102 can measure data (e.g., raw measurement data, milk characteristic information determined based on raw measurement data) during a milking session that can be used to determine milk quality metrics. At 510, sensors 102 can transmit data (e.g., raw measurement data, milk characteristic information determined based on raw measurement data) to livestock management system 502 (e.g., via a local wireless connection, via a dedicated wired connection, via a LAN, via a WAN, etc.). As described above, a livestock management system can be implemented locally and/or remotely, and accordingly, transmission of data by sensors 102 at 510 can include transmission to a local device (e.g., a local computing device, such as local computing device 220) or a remote device (e.g., a dedicated remote server, a cloud computing service, etc.).

At 512, livestock management system 502 can receive data generated by sensors 102, and at 514, livestock management system 502 can associate the sensor data with a particular animal (e.g., based on a determination that the particular animal was milked using a milking station associated with the sensor while the data was being generated).

At 516, livestock management system 502 can determine milk composition information for the milking session corresponding to the data received at 512. For example, livestock management system 502 can use techniques described above in connection with 408 of FIG. 4 to determine milk composition information for the milking session.

At 518, livestock management system 502 can determine milk quality information that is based on data received for multiple milking sessions (e.g., likely from different sensors 102 as the cow is milked at different milking stations). For example, livestock management system 502 can use techniques described above in connection with 410 of FIG. 4 to determine milk quality information based on data for multiple milking sessions.

At 520, livestock management system 502 can provide access to milk quality information (e.g., milk quality information determined at 518) for each animal being monitored and/or for one or more groups of animals (e.g., a subset of cows in a herd that share a particular characteristic). For example, livestock management system 502 can store milk quality information, and an authorized user can be permitted to access the stored milk quality information using a user computing device (e.g., via a webpage that facilitates access to data stored by livestock management system 502, via an installed application such as a mobile application that that facilitates access to data stored by livestock management system 502, via an authorized application program interface (API) call from a computing device, etc.). At 522, user computing device 504 can present milk quality information accessed from livestock management system 502 via a user interface (e.g., a graphical user interface). In some embodiments, flow 500 can omit 520 and 522, for example where a user permits livestock management system 502 to initiate adjustments to one or more variables associated with care of a particular animal autonomously (note, however, that users generally can be permitted to access the milk quality information, regardless of whether adjustments are carried out autonomously).

At 524, livestock management system 502 can determine an adjustment to a variable associated with a particular animal based on milk quality information (e.g., as described above in connection with 416 of FIG. 4). For example, livestock management system 502 can determine an adjustment that is likely to: improve the health of the animal; maintain the health of the animal from deteriorating; improve milk production by the animal; etc.). In some embodiments, livestock management system 502 can prompt a user to review and/or authorize the adjustment (e.g., as described above in connection with 416 of FIG. 4). In such embodiments, livestock management system 502 can cause a user computing device (e.g., user computing device 504) to present information about the adjustment determined at 524 (e.g., by transmitting an alert via a push notification to a user computing device(s) associated with a particular user(s), a text message, an email, etc.). In some embodiments, flow 500 can omit 524, for example where a user does not permit livestock management system 502 to suggest adjustments to one or more variables associated with care of animals autonomously.

At 526, user computing device 504 can present information about an adjustment determined at 524 (e.g., via a graphical user interface). In some embodiments, the information about the adjustment can include any suitable information, such as identifying information of the animal for which the adjustment is to be made, information indicating what prompted the adjustment, at least a portion of the milk quality information for the animal(s) and/or a summary of milk quality information for the animal(s), information describing the proposed adjustment, etc.

At 528, user computing device 504 can receive input to adjust one or more variables associated with a particular animal(s) (e.g., via a GUI). For example, if livestock management system 502 generated a suggested adjustment at 524 that requires authorization, input can be provided at 528 to cause the adjustment to be implemented. As another example, regardless of whether livestock management system 502 generated a suggested adjustment at 524, a user may be permitted to provide input to cause an adjustment to be implemented for one or more animals (e.g., a user can implement adjustments if livestock management system 502 is not configured to automatically determine adjustments and/or if the user desires to make an adjustment that has not been identified by livestock management system 502). In some embodiments, flow 500 can omit 526 and/or 528, for example where a user permits livestock management system 502 to initiate adjustments to one or more variables associated with care of a particular animal autonomously (note, however, that users generally can be permitted to access the milk quality information, regardless of whether adjustments are carried out autonomously).

At 530, livestock management system 502 can instruct one or more automated farm equipment systems to implement an adjustment to the variable(s) determined at 524 and/or input at 528. At 532, automated farm equipment 506 can receive an instruction(s) to adjust a variable(s) for a particular animal(s) from livestock management system 502 and/or user computing device 504. At 534, automated farm equipment 506 can alter conditions for the animal based on the instruction (e.g., by adjusting the amount and/or composition of feed provided to the animal).

### FURTHER EXAMPLES HAVING A VARIETY OF FEATURES

Implementation examples are described in the following numbered clauses:

1. A method for automated milk quality monitoring using inline sensors, the method comprising: receiving, from each of a plurality of inline sensors, data indicative of milk composition information of milk flowing through that inline sensor during a milking session, wherein each of the plurality of inline sensors is configured to measure milk composition information of milk flowing through one of a plurality of milk lines, which are each associated with a respective milking station of a plurality of milking stations, wherein a first subset of milk lines of the plurality of milk lines are equipped with a respective inline sensor of the plurality of inline sensors, such that milk composition information is generated for milk flowing through the first subset of milk lines during milking, and wherein a second subset of milk lines the plurality of milk lines are not equipped with inline an inline sensor configured to measure milk composition information of milk flowing through one of the second subset of milk lines, such that milk composition information is not generated for milk flowing through the second subset of milk lines during milking; associating the received data with a particular animal that produced the milk; storing milk composition information for milk from the particular animal during the milking session; and determining milk quality information for the particular animal using stored milk composition information for a plurality of milking sessions over a predetermined period of time.

2. The method of clause 1, wherein the number of milk lines in the first subset of milk lines is less than the number of milk lines in the second subset of milk lines.

3. The method of clause 2, wherein each milk line of the plurality of milk lines is a member of the first subset of milk lines of the second subset of milk lines.

4. The method of any one of clauses 1 to 3, wherein each of the plurality of inline sensors comprises: a laser source configured to emit coherent light through milk flowing through the inline sensor; and a laser detector configured to generate spectral data indicative of the concentration of one or more milk components in the milk through which the coherent light passed.

5. The method of clause 4, wherein each of the plurality of inline sensors comprises: a contact sensor configured to determine a volumetric flow of milk through the sensor.

6. The method of any one of clauses 1 to 5, further comprising: receiving, from each of a plurality of inline volume sensors configured to measure volume flow of milk flowing through one of the plurality of milk lines, data indicative of volumetric flow, wherein each milk line in the first subset of milk lines is equipped with a respective inline volume sensor of the plurality of inline volume sensors, such that milk volume information is generated for milk flowing through the first subset of milk lines during milking.

7. The method of any one of clauses 5 or 6, further comprising: receiving data indicative of volumetric flow of milk flowing through a particular milk line during the milking session; determining the milk composition information for milk from a particular animal during a particular milking session based on the data indicative of milk composition information of milk flowing through the particular milk line during the milking session and the data indicative of volumetric flow of milk flowing through the particular milk line during the milking session.

8. The method of any one of clauses 1 to 7, wherein the plurality of milking stations comprise all of the milk stations of a milking operation, such that only a fraction of milking stations of the milking operation are equipped with inline sensors configured to measure milk composition information of milk during milking.

9. The method of clause 8, wherein the first subset of milk lines is about ten percent of the plurality of milking stations.

10. The method of clause 8, wherein the milking operation comprises a robotic system configured to autonomously attach milking devices to animals at the plurality of milking stations for milking.

11. The method of any one of clauses 1 to 10, wherein the stored milk composition information comprises fat content information for the milking session, protein content information for the milking session, and lactose content information for the milking session.

12. The method of any one of clauses 1 to 11, wherein the milk quality information is an aggregate value based on milk quality information for each of the plurality of milking sessions.

13. The method of any one of clauses 1 to 12, wherein the predetermined period of time is at least seven days.

14. A system comprising: at least one processor that is configured to: perform a method of any of clauses 1 to 13.

15. A non-transitory computer-readable medium storing computer-executable code, comprising code for causing a computer to cause a processor to: perform a method of any of clauses 1 to 13.

In some embodiments, any suitable computer readable media can be used for storing instructions for performing functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

It should be noted that, as used herein, the term mechanism can encompass hardware, software, firmware, or any suitable combination thereof.

It should be understood that above-described steps of the process of FIG. 4 can be executed or performed in any suitable order or sequence not limited to the order and sequence shown and described in the figures. Also, some of the above steps of the process of FIG. 4 can be executed or performed substantially simultaneously where appropriate or in parallel to reduce latency and processing times.

This written description uses examples to disclose the invention(s), including the best mode, and also to enable any person skilled in the art to make and use the invention(s). Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention(s) is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

What is claimed is:

1. A system for automated milk quality monitoring using inline sensors, the system comprising:
a plurality of milking stations, each associated with a respective milk line of a plurality of milk lines;
a plurality of inline sensors, each configured to measure milk composition information of milk flowing through one of the plurality of milk lines,
wherein a first subset of milk lines of the plurality of milk lines are equipped with a respective inline sensor of the plurality of inline sensors, such that milk composition information is generated for milk flowing through the first subset of milk lines during milking, and
wherein a second subset of milk lines the plurality of milk lines are not equipped with inline an inline sensor configured to measure milk composition information of milk flowing through one of the second subset of milk lines, such that milk composition information is not generated for milk flowing through the second subset of milk lines during milking; and
one or more hardware processors configured to:
receive, from each of the plurality of inline sensors, data indicative of milk composition information of milk flowing through that inline sensor during a milking session;
associate the received data with a particular animal that produced the milk;
store milk composition information for milk from the particular animal during the milking session; and
determine milk quality information for the particular animal using stored milk composition information for a plurality of milking sessions over a predetermined period of time.

2. The system of claim 1, wherein the number of milk lines in the first subset of milk lines is less than the number of milk lines in the second subset of milk lines.

3. The system of claim 2, wherein each milk line of the plurality of milk lines is a member of the first subset of milk lines of the second subset of milk lines.

4. The system of claim 1, wherein each of the plurality of inline sensors comprises:
a laser source configured to emit coherent light through milk flowing through the inline sensor; and
a laser detector configured to generate spectral data indicative of the concentration of one or more milk components in the milk through which the coherent light passed.

5. The system of claim 4, wherein each of the plurality of inline sensors comprises:
a contact sensor configured to determine a volumetric flow of milk through the sensor.

6. The system of claim 1, further comprising:
a plurality of inline volume sensors, each configured to measure volume flow of milk flowing through one of the plurality of milk lines,
wherein each milk line in the first subset of milk lines is equipped with a respective inline volume sensor of the plurality of inline volume sensors, such that milk volume information is generated for milk flowing through the first subset of milk lines during milking.

7. The system of claim 6, wherein the one or more hardware processors are further configured to:
receive data indicative of volumetric flow of milk flowing through a particular milk line during the milking session; and
determine the milk composition information for milk from a particular animal during a particular milking session based on the data indicative of milk composition information of milk flowing through the particular milk line during the milking session and the data indicative of volumetric flow of milk flowing through the particular milk line during the milking session.

8. The system of claim 1, wherein the plurality of milking stations comprise all of the milk stations of a milking operation, such that only a fraction of milking stations of the milking operation are equipped with inline sensors configured to measure milk composition information of milk during milking.

9. The system of claim 8, wherein the first subset of milk lines is about ten percent of the plurality of milking stations.

10. The system of claim 8, wherein the milking operation comprises a robotic system configured to autonomously attach milking devices to animals at the plurality of milking stations for milking.

11. The system of claim 1, wherein the stored milk composition information comprises fat content information for the milking session, protein content information for the milking session, and lactose content information for the milking session.

12. The system of claim 1, wherein the milk quality information is an aggregate value based on milk quality information for each of the plurality of milking sessions.

13. The system of claim 1, wherein the predetermined period of time is at least seven days.

14. A method for automated milk quality monitoring using inline sensors, the method comprising:
receiving, from each of a plurality of inline sensors, data indicative of milk composition information of milk flowing through that inline sensor during a milking session,
wherein each of the plurality of inline sensors is configured to measure milk composition information of milk flowing through one of a plurality of milk lines, which are each associated with a respective milking station of a plurality of milking stations,
wherein a first subset of milk lines of the plurality of milk lines are equipped with a respective inline sensor of the plurality of inline sensors, such that milk composition information is generated for milk flowing through the first subset of milk lines during milking, and
wherein a second subset of milk lines the plurality of milk lines are not equipped with inline an inline sensor configured to measure milk composition information of milk flowing through one of the second subset of milk lines, such that milk composition information is not generated for milk flowing through the second subset of milk lines during milking;
associating the received data with a particular animal that produced the milk;
storing milk composition information for milk from the particular animal during the milking session; and
determining milk quality information for the particular animal using stored milk composition information for a plurality of milking sessions over a predetermined period of time.

15. The method of claim 14, wherein the number of milk lines in the first subset of milk lines is less than the number of milk lines in the second subset of milk lines.

16. The method of claim 15, wherein each milk line of the plurality of milk lines is a member of the first subset of milk lines of the second subset of milk lines.

17. The method of claim 14, wherein each of the plurality of inline sensors comprises:
a laser source configured to emit coherent light through milk flowing through the inline sensor; and
a laser detector configured to generate spectral data indicative of the concentration of one or more milk components in the milk through which the coherent light passed.

18. The method of claim 17, wherein each of the plurality of inline sensors comprises:
a contact sensor configured to determine a volumetric flow of milk through the sensor.

19. The method of claim 14, further comprising:
receiving, from each of a plurality of inline volume sensors configured to measure volume flow of milk flowing through one of the plurality of milk lines, data indicative of volumetric flow,
wherein each milk line in the first subset of milk lines is equipped with a respective inline volume sensor of the plurality of inline volume sensors, such that milk volume information is generated for milk flowing through the first subset of milk lines during milking.

20. The method of claim 19, further comprising:
receiving data indicative of volumetric flow of milk flowing through a particular milk line during the milking session; and
determining the milk composition information for milk from a particular animal during a particular milking session based on the data indicative of milk composition information of milk flowing through the particular milk line during the milking session and the data indicative of volumetric flow of milk flowing through the particular milk line during the milking session.
